SEQUENCE LISTING (2 sequences)

SEQ ID NO: 1 - protein, 56 aa, P. pastoris
  EPI-HNE4: N-terminal modified to facilitate secretion
EACNLPIVRGPCIAFFPRWAFDAVKGKCVLFPYGGCQGNGNKFYSEKECREYCGVP

SEQ ID NO: 2 - protein, 58 aa, P. pastoris
  ITI-D2: Second Kunitz domain of the light chain of human inter-alpha inhibitor protein
TVAACNLPIVRGPCRAFIQLWAFDAVKGKCVLFPYGGCQGNGNKFYSEKECREYCGVP

(19) United States
(12) Patent Application Publication
(10) Pub. No.: US 2009/0074843 A1
(43) Pub. Date: Mar. 19, 2009

(54) Use of epi-hne 1-4

(76) Inventors: Flemming Reissig Jensen, Vaerloese (DK); Lene Karin Jespersen, Fredensborg (DK); Sanne Lise Lauemoeller, Helsinge (DK); Kristian Larsen, Vaerloese (DK); Chaabane Bougherara, Frederiksberg (DK)

Correspondence Address: JACOBSON HOLMAN PLLC, 400 SEVENTH STREET N.W., SUITE 600, WASHINGTON, DC, 20004, US

(21) Appl. No.: 11/547,167

(22) PCT Filed: Mar. 30, 2005
(86) PCT No.: PCT/DK2005/000216

§ 371 Date: Sep. 15, 2008

(30) Foreign Application Priority Data
Mar. 31, 2004 (DK) PA 2004 00511

(51) Int. Cl. A61L 15/44; C07K 14/47

(52) U.S. Cl. 424/445; 530/324

(57) ABSTRACT

The invention relates to use of at least one of the compounds EPI-HNE 1, EPI-HNE 2, EPI-HNE 3 or EPI-HNE 4 for the manufacture of a medicament for treatment of chronic wounds or burns. The EPI-HNE 1-4 compounds are e.g. suitable for incorporation into a wound care device, such as a dressing.

This is a nationalization of PCT/DK05/000216 filed Mar. 30, 2005 and published in English.

### BACKGROUND OF THE INVENTION

1. Field of the Invention

The invention relates to use of Epihne4 for the manufacture of a medicament, especially for treatment of chronic wounds.

The world has seen a dramatic increase in number of elderly people, an increase that is estimated to continue in the future. It is estimated that the world's elderly population (>65) will grow from approximately 500 millions in 1997 to more than one billion in 2030. The increase is driven by an increased life expectancy in general combined with a low birth rate in the developed world.

Chronic wounds typically affect people over 65, due to the underlying clinical complications typically associated with chronic wounds, such as venous and arterial insufficiency, diabetes and trauma, and pressure sores caused by bedrest. Besides the economic aspects of chronic wound treatment, the patients are suffering tremendously from a combination of infections, pain and low quality of life, combined with a significant increased risk of amputation of limbs and premature death.

Current treatment of chronic wounds varies across the world, advanced wound healing devices based on passive bandages facilitating moist wound healing and exudate control being the current state of the art. It is therefore essential that efficient treatment of chronic wounds be developed, in particular treatments based on the active manipulations of problematic molecular factors in the wound contributing to chronicity through actively retarding the healing process.

2. Description of the Related Art

Serine Proteases are proteolytic enzymes naturally occurring in humans. This family of enzymes has many functions in the human physiology, e.g. in the immune system, foetal development, cancer and inflammatory pathways.

Human Neutrophil Elastase (HNE, synonyms: Elastase, leukocyte elastase, lysosomal elastase) is a pro-inflammatory Serine Protease, and is one of several proteolytic enzymes contained in the azurophil granules of human neutrophils. Elastase is involved in the inflammatory response in general including wounding, and as such, Elastase is involved in the degradation of foreign material ingested during phagocytosis, as well the degradation of extracellular matrix components such as Collagen, Fibronectin and Elastin.

It has been demonstrated that levels of elastase activity are elevated in chronic wound fluids and in burns and that elastase contributes to the overall increase in proteolytic activity of the chronic wound environment.

Elastase, present in chronic wound fluid, is the enzyme responsible for the degradation of several extracellular constitutive matrix proteins such as Fibronectin, Collagen and Elastin, and this excessive proteolytic activity results in undesirable degradation of the extracellular matrix necessary for re-epitheliazation of the wound bed. The resulting matrix protein fragments are neutrophilic chemoattractants, enhancing the recruitment of neutrophils increasing the inflammatory burden on the already inflamed area.

Elastase is also involved in the degradation of peptide growth factors such as PDGF and TGF-β, which are considered to be necessary for the healing to occur, and cell surface receptors for peptide growth factors may themselves be functionally inactivated by the actions of elastase.

Elastase is also known to activate pro-inflammatory Matrix Metallo Proteases (MMP's), leading to increased protease activity and further catabolic tissue damage.

Under normal conditions, Elastase is controlled by naturally occurring inhibitors, such as SLPI, AAT or Elafin, serum protease inhibitors, which can penetrate into various tissues. However, studies have shown that the level of α-1-antitrypsin is low in chronic wounds compared to acute wounds, which may contribute to the non-healing of persistent chronic wounds.

Protease inhibitors normally prevent damage to connective tissue caused by leakage of MMP's, however elastase is known to proteolytically inactivate naturally occurring specific MMP-inhibitors, TIMP's. Furthermore, MMP's have been demonstrated to degrade AAT. In addition, elastase itself may participate in proteolytic activation of collagenase and gelatinase, contributing to undesirable proteolytic activity in the chronic wound environment.

Several attempts have been made to utilize AAT or SLPI as an elastase inhibiting anti-inflammatory agent:

WO 99/49887 describes a method of treating wounds by topically administering an effective amount of a serine protease inhibitor to a wound. The claimed invention specifically relates to urokinase inhibitors.

GB 2318732 discloses the use of AAT for the preparation of a composition for the treatment of a chronic wound.

WO 01/64031 relates to a method for treating a wound comprising administering a wound-healing dose of SLPI.

However, the chronic wound environment is extremely hostile to these inhibitors, causing inactivation by a multitude of mechanisms, as demonstrated below.

One problem is that SLPI and AAT loose anti-elastase activity when exposed to reactive oxygen species (ROS), due to oxidation of the active site methionine to the corresponding sulfoxide. Reactive oxygen species are known to exist in high concentrations in chronic wound exudates as part of the excessive inflammatory response. Furthermore, native AAT and SLPI are substrates for other proteases known to be elevated in chronic wounds, primarily Matrix Metallo Proteases (MMP's) e.g. Stromelysin, MMP-3, rendering the enzymes cleaved and inactive. All of these factors contribute to reduced half-life and low efficacy in vivo.

Oxygenated AAT is furthermore susceptible to degradation by elastase, the degradation fragments being chemoattractants towards neutrophils.

Another disadvantage of controlling elastase by means of AAT is that metabolites of AAT are suspected to be involved in certain pro-inflammatory processes. For example ox-AAT is suspected to be pro-inflammatory through monocyte activation, leading to increased concentration of inflammatory mediators such as TNF-alfa, IL6, MMP-1 and MMP-9. Cleavage fragments from both native AAT (C-36 fragment) as well as from ox-AAT have been suggested to have a similar monocyte activating effect.

Lastly, yet another problem associated with the administration of AAT is that the complex between AAT and elastase arising from AAT inhibition of the protease is also suspected to have pro-inflammatory properties through a chemotactic effect on neutrophils. A study of an inhalable form of AAT have shown the AAT-elastase complex diffuses into the lung interstitium where the complex dissociates due to the low Ki of AAT, thereby releasing active elastase with a catabolic result.

Thus there is still a need for a wound care device for treatment of chronic wounds and burns, which is capable of inhibiting elastase and thereby enhancing the healing process of such wounds.

### SUMMARY OF THE INVENTION

The object of the present invention is to facilitate accelerated healing of chronic wounds and burns by improving tissue regeneration.

Another object of the invention is to reduce the inflammation in chronic wounds/burns.

Yet another object of the present invention is to provide a faster healing of chronic wounds and burns by inhibiting the elastase activity in the wounds or burns.

Another object of the invention is to administer to the wound an elastase inhibitor, which is not susceptible to degradation by the proteolytic environment of chronic wounds/burns or to oxidation in the wound bed/wound exudate.

Still another object of the invention is to provide an elastase inhibitor, itself nor alterations thereof (degradations products, oxidation products) not being pro-inflammatory, thereby eliminating the risk of increasing the inflammation already present in the chronic wound/burn.

A further object of the invention is to provide a wound dressing comprising an elastase inhibitor, being sturdy and stable during sterilisation, storage and exposure to wound exudates.

Yet another object of the invention is to provide a dressing having a prolonged wear time.

### DETAILED DESCRIPTION OF THE PRESENT INVENTION

The invention relates to use of at least one of the compounds EPI-HNE 1, EPI-HNE 2, EPI-HNE 3 or EPI-HNE 4 for the manufacture of a medicament for treatment of chronic wounds.

The invention further relates to the use of at least one of the compounds EPI-HNE 1, EPI-HNE 2, EPI-HNE 3 or EPI-HNE 4 for the manufacture of a medicament for treatment of burns.

EPI-HNE 1-4 are all compounds capable of inhibiting human neutrophil elastase, they all have remarkably similar abilities to inhibit HNE-routes to other HNE-inhibitory sequences.

It is to be understood that the term EPI-HNE 1-4 as used herein encompasses functional fragments of EPI-HNE 1-4, chimeric proteins comprising EPI-HNE 1-4 or functional fragments thereof, homologs obtained by analogous substitution of one or more amino acids of EPI-HNE 1-4, and species homologs. Furthermore, functional terminal modifications in general and peptide chain extensions especially are covered, e.g. the attachment of up to a 10 amino acids peptide fragment N-terminally on EPI-HNE 1-4.

In a preferred embodiment of the present invention, the active ingredient is EPI-HNE4, a 56 amino acid protein (6231 Da, EPI-HNE4 amino acid sequence is given below), discovered using a technology called “Directed evolution of novel binding proteins”, is derived from the second Kunitz type domain of the light chain of the human inter-alpha-inhibitor protein (ITI-D2) [U.S. Pat. No. 5,663,143, and references herein, hereafter incorporated in its entirety as reference].

The sequence below is aligned to the parental domain (ITI-D2) based on the 6 cysteines characteristic of the Kunitz type domain from which EPI-HNE4 is derived, in such a way that the first cysteine is assigned position 5.

EPI-HNE4 is member of a family of potent elastase inhibitors. EPI-HNE4 has been modified in the N-terminal residue to facilitate secretion from the yeast species P. pastoris, in which it can be produced by fermentation.

EPIHNE4:
(SEQID NO: 1)
EACNLPIVRGPCIAFFPRWAFDAVKGKCVLFPYGGCQGNGNKFYSEKECREYCGVP
ITI-D2:
(SEQID NO: 2)
TVAACNLPIVRGPCRAFIQLWAFDAVKGKCVLFPYGGCQGNGNKFYSEKECREYCGVP

It has surprisingly been shown that by administering EPI-HNE1-4 to a wound, an efficient elastase inhibition may be obtained and stability problems may be reduced.

EPI-HNE 1-4 have been shown to be resistant to oxidation using chloramineT (up to 50 mol-equivalents), conditions which destroy the activity of both AAT and SLPI. EPI-HNE 1-4 are also able to tolerate relatively high temperatures for up to 18 hours at pH 7 (65° C. for 18 hours at pH 7). Furthermore, EPI-HNE 1-4 are known to be resistant to proteolytic degradation.

It has been suggested that, in a cystic fibrosis context, an elastase inhibitor, in order to be an efficient therapeutic agent, must have a K_{D}-value below 0.1 nanoM. EPI-HNE 1-4 are very potent inhibitors of human neutrophil elastase, having a K_{D} of 4 picoM. For comparison, the corresponding K_{D} values for AAT is 10⁻⁷ M, and for SLPI 10⁻⁷ M.

Furthermore, EPI-HNE 1-4 has been synthetically prepared and thus does not suffer from the problems that may arise when using AAT, which has been extracted from plasma, and compared to yeast-prepared AAT; the EPI-HNE 1-4 may have a longer half-life. Thus, a lower dose may be sufficient and/or wear time may be prolonged.

In the therapeutic use of these potent active inhibitors, it is always extremely important to avoid potential side effects from the administration of the therapeutics. EPI-HNE4 was tested against a number of human household enzymes [U.S. Pat. No. 5,663,143] such as Human Serum Plasmin, Kallikrein and Thrombin as well as Human Urine Urokinase, Human Plasma Factor Xₐ and Human Pancreatic Chymotrypsin. In all cases a selectivity in Kᵢ values of more than 10⁶ was observed, i.e., the activity of EPI-HNE4 is 10⁶ times lower towards the mentioned household enzymes than towards Elastase.

No evidence exist to suggest that EPI-HNE 1-4, elastase-EPI-HNE 1-4 complex or EPI-HNE 1-4 metabolites are pro-inflammatory, and therefore, combined with the extremely low K_{D} of low picomolar range, it has surprisingly been shown that EPI-HNE 1-4 are superior agents for the treatment of chronic wounds and burns, exceeding by several orders of magnitude the potency of AAT and SLPI towards elastase.

Furthermore, EPI-HNE1-4 have properties ensuring a superior stability in the hostile chronic wound environment as well as no pro-inflammatory mediation from metabolites or complexes has been shown. Therefore, EPI-HNE4 is claimed to be a superior agent for use in the treatment of chronic wounds.

In a preferred embodiment of the invention the invention relates to the use of EPI-HNE4 for the manufacture of a medicament for treatment of chronic wound.

In another preferred embodiment of the invention the invention relates to the use of EPI-HNE4 for the manufacture of a medicament for treatment of burns.

It is preferred that the treatment of the wounds or burns is local or topical, but a systemic treatment may also be used instead or in combination with the local/topical use.

The medicament may be incorporated in a wound dressing or it may be administered locally or topically to the wound or burn. The medicament may be formulated as a gel or cream or ointment, or it may be released from a wound dressing, optionally through a controlled or sustained release profile matching clinical needs and dressing wear time.

The invention further relates to a dressing for treatment of chronic wounds or burns, wherein said dressing comprises at least one elastase inhibitor selected from the group of EPI-HNE 1, EPI-HNE 2, EPI-HNE 3 or EPI-HNE 4. In a preferred embodiment of the invention the elastase inhibitor is EPI-HNE 4.

The dressing may further comprise an absorbent element, and/or it may comprise a backing layer. The backing layer may preferably be water impervious but vapor permeable. The dressing may further comprise an adhesive, such as a hydro-colloid adhesive. In one embodiment of the invention the EPI-HNE 1-4 may be incorporated in the adhesive.

In one embodiment of the invention the dressing comprises a gel. The EPI-HNE1-4 may be incorporated into a vehicle. Such vehicle may, although not to be considered as limiting for the invention, be selected from the group of a synthetic polymer material, such as a foam matrix (such as polyurethane foam), polymer beads (such as PEG-beads or PEG sheets), bio-polymers (such as hyaluronic acid, alginates, chitosans, Collagen), liposomes and coated particles. In one embodiment of the invention the laminate of the invention may comprise one or more active ingredients.

In embodiment of the invention the dressing of the invention may comprise one or more active ingredients together the EPI-HNE 1-4.

The active ingredient may also comprise odor controlling or odor reducing material such as charcoal.

The active ingredient may be present in dressing, but not necessarily in direct contact with the skin or wound, or it may migrate to the wound when exposed to moisture, such as wound exudate.

It is advantageous to provide the dressing of the invention with components for treatment or prophylaxis of formation of wounds and/or skin abnormalities, e.g. with emollients or an active constituent e.g. retinoids for treating or preventing formation of psoriasis, eczema, callous skin, corns, insect bites, acne or blisters. The dressing of the invention may also contain medicaments such as bacteriostatic or bactericide compounds, e.g. iodine, iodopovidone complexes, chloramine, chlorohexidine, silver salts, zinc or salts thereof, tissue-healing enhancing agents, e.g. RGD tripeptides and the like, enzymes for cleansing of wounds, e.g. pepsin, trypsin, papain and the like, pain relieving agents, such as ibuprofen, ketoprofen etc., or agents having a cooling effect which is also considered an aspect of the invention.

Such agents may be incorporated into the dressing e.g. be enclosed in the adhesive or in an absorbent layer.

The invention further relates to a method of treatment of a chronic wound or burns, wherein at least one elastase inhibitor selected from the group of EPI-HNE 1, EPI-HNE 2, EPI-HNE 3 or EPI-HNE 4 is administered to a wound or burn in an effective amount.

1. Use of at least one of the compounds EPI-HNE 1, EPI-HNE 2, EPI-HNE 3 or EPI-HNE 4 for the manufacture of a medicament for treatment of chronic wounds.

2. Use of at least one of the compounds EPI-HNE 1, EPI-HNE 2, EPI-HNE 3 or EPI-HNE 4 for the manufacture of a medicament for treatment of burns.

3. Use according to claim 1 of EPI-HNE4 for the manufacture of a medicament for treatment of chronic wound.

4. Use according to claim 1 of EPI-HNE4 for the manufacture of a medicament for treatment of burns.

5. Use according to claim 1 wherein the medicament is incorporated in a wound dressing.

6. A dressing for treatment of chronic wounds or burns, wherein said dressing comprises at least one elastase inhibitor selected from the group of the compounds EPI-HNE 1, EPI-HNE 2, EPI-HNE 3 or EPI-HNE 4.

7. A dressing according to claim 6 wherein the elastase inhibitor is EPI-HNE 4.

8. A dressing according to claim 6, wherein the dressing comprises an absorbent element.

9. A dressing according to claim 6, wherein at least one of the compounds EPI-HNE 1, EPI-HNE 2, EPI-HNE 3 or EPI-HNE 4 is incorporated into a vehicle.

10. Method of treatment of a chronic wound or burns, wherein at least one elastase inhibitor selected from the group of the compounds EPI-HNE 1, EPI-HNE 2, EPI-HNE 3 or EPI-HNE 4 is administered to a wound or burn in an effective amount.

11. A dressing according to claim 7, wherein the dressing comprises an absorbent element.

12. A dressing according to claim 7 wherein at least one of the compounds EPI-HNE 1, EPI-HNE 2, EPI-HNE 3 or EPI-HNE 4 is incorporated into a vehicle.

13. A dressing according to claim 8 wherein at least one of the compounds EPI-HNE 1, EPI-HNE 2, EPI-HNE 3 or EPI-HNE 4 is incorporated into a vehicle.
